(19) United States
(12) Patent Application Publication
(10) Pub. No.: US 2017/0067025 A1
(43) Pub. Date: Mar. 9, 2017

(54) TUMOR MODEL FOR BREAST CANCER CELL MIGRATION STUDIES AND RELATED METHODS

(71) Applicant: ARIZONA BOARD OF REGENTS ON BEHALF OF ARIZONA STATE UNIVERSITY, Scottsdale, AZ (US)

(72) Inventors: Mehdi Nikkhah, Scottsdale, AZ (US); Feba Sam, Mumbai (IN); Nitish Peela, Chandler, AZ (US)

(21) Appl. No.: 15/252,777

(22) Filed: Aug. 31, 2016

Related U.S. Application Data
(60) Provisional application No. 62/213,468, filed on Sep. 2, 2015.

(51) Int. Cl. C12N 5/09; G03F 7/20; C12N 5/00

(52) U.S. Cl. CPC C12N 5/0693; C12N 5/0012; G03F 7/70; C12N 2537/10; C12N 2535/00; C12N 2533/30; C12N 2531/00

(57) ABSTRACT

A method for creating a tumor model includes encapsulating cancer cells in a first solution, disposing the first solution on a spacer, cross-linking the first solution and creating one or more high stiffness constructs, disposing a second solution around the one or more high stiffness constructs, and cross-linking the second solution and creating a low stiffness matrix surrounding the one or more low stiffness constructs.

### CROSS-REFERENCE TO RELATED APPLICATION

This application claims priority from U.S. Provisional Application No. 62/213,468, filed Sep. 2, 2015. The entire content of this provisional application is hereby incorporated herein by reference.

### BACKGROUND

Metastatic dissemination of cancer cells is a highly complex and multi-step biological process starting with tumor angiogenesis and the invasion of cancer cells through the extracellular (ECM) matrix toward the blood vessels. Cancer cell invasion through the tumor stroma is governed by diverse factors including biochemical signals and biophysical cues. Despite their significance, most in vivo animal models present an abundance of confounding variables making it challenging to attribute specific microenvironmental cues to cellular invasion. In this regard, physiologically relevant in vitro tumor models are crucial to understand cancer cell invasion within a native-like breast tumor microenvironment.

In the past few years, there has been a tremendous initiative to develop in vitro models to study cancer cell behavior in 3D microenvironments. For instance, three-dimensional (3D) surface topographies have been widely used to study cancer cell behavior in response to various geometrical features. Despite their significance, these platforms lacked the capacity for varying parameters including stiffness and matrix architecture. Alternatively, a wide variety of 3D hydrogel-based matrices such as Matrigel, fibrin, collagen, and polyethylene glycol (PEG) have shown great promise to recapitulate cancer cell invasion in a 3D matrix and assess cellular behavior in response to various biophysical and biochemical cues. Such 3D hydrogel-based matrices enable cells to retain accurate phenotype and, consequently, exhibit precise responses to microenvironmental stimuli along with cell-cell and cell-matrix interactions. These models, however, lack specific patterned features that would enable precise control over cellular distribution and matrix stiffness to conduct studies within biomimetic tumor architecture.

### SUMMARY

In one or more embodiments, a method for creating a tumor model includes encapsulating cancer cells in a first solution, disposing the first solution on a spacer, cross-linking the first solution and creating one or more high stiffness constructs, disposing a second solution around the one or more high stiffness constructs, and cross-linking the second solution and creating a low stiffness matrix surrounding the one or more low stiffness constructs.

In one or more embodiments, encapsulating the cancer cells includes encapsulating breast cancer cells in GelMA prepolymer solution.

In one or more embodiments, cross-linking the first solution includes conducting a first photolithography session on the first solution.

In one or more embodiments, cross-linking the second solution includes conducting a second photolithography session on the second solution.

In one or more embodiments, disposing a second solution around the one or more high stiffness constructs includes disposing pristine GelMA prepolymer solution and spreading the pristine GelMA prepolymer solution between the high stiffness constructs.

In one or more embodiments, creating one or more high stiffness constructs includes creating an array of high stiffness constructs.

In one or more embodiments, cross-linking the first solution includes exposing the first solution to UV light.

In one or more embodiments, cross-linking the second solution includes exposing the second solution to UV light.

In one or more embodiments, creating one or more high stiffness constructs includes creating circular high stiffness constructs.

In one or more embodiments, a method includes conducting a first photolithography session including encapsulating breast cancer cells in GelMA prepolymer solution, disposing the GelMA prepolymer solution on a spacer, exposing the GelMA prepolymer solution and encapsulated breast cancer cells to UV light to crosslink GelMA and creating one or more high stiffness constructs on a first slide, and removing the first slide from the spacer; and

conducting a second photo lithography session including disposing pristine GelMA prepolymer solution on to the spacer, disposing the first slide over the pristine GelMA and spreading the pristine GelMA prepolymer solution between the high stiffness circular constructs, and exposing the second assembly to UV light to crosslink the GelMA.

In one or more embodiments, exposing the second assembly and crosslinking the pristine GelMA includes creating a low stiffness matrix surrounding the high stiffness constructs.

In one or more embodiments, creating one or more high stiffness constructs includes creating an array of high stiffness constructs.

In one or more embodiments, creating one or more high stiffness constructs includes creating circular high stiffness constructs.

In one or more embodiments, a method for creating a tumor model comprises encapsulating breast cancer cells in GelMA prepolymer solution, disposing the GelMA prepolymer solution on a spacer, disposing a photomask and glass slide on the spacer to form a first assembly, exposing the first assembly to UV light to crosslink GelMA and creating an array of high stiffness constructs on a first slide. The method further includes removing the first slide from the spacer, disposing pristine GelMA prepolymer solution on to the spacer, disposing the first slide over the pristine GelMA and spreading the pristine GelMA prepolymer solution between the high stiffness constructs to form a second assembly, and exposing the second assembly to UV light to crosslink the GelMA.

In one or more embodiments, exposing the second assembly and crosslinking the pristine GelMA includes creating a low stiffness matrix surrounding the high stiffness constructs.

In one or more embodiments, creating the array of high stiffness constructs includes creating an array of circular high stiffness constructs.

In one or more embodiments, a tumor model comprises a first slide having a first region of high stiffness constructs, the high stiffness constructs including cancer cells encapsulated therein, where the first slide includes a second region of low stiffness matrix surrounding the high stiffness constructs, and the first region has a higher stiffness than the second region.

In one or more embodiments, the high stiffness constructs are micropatterned circular constructs.

In one or more embodiments, the high stiffness constructs include crosslinked GelMA.

### BRIEF DESCRIPTION OF THE DRAWINGS

FIG. 1A illustrates a schematic diagram including a portion of a first photolithography session, in accordance with one or more embodiments.

FIG. 1B illustrates a schematic diagram including a portion of a first photolithography session in accordance with one or more embodiments.

FIG. 1C illustrates a schematic diagram including a portion of a first photolithography session in accordance with one or more embodiments.

FIG. 1D illustrates a schematic diagram including a portion of a second photolithography session in accordance with one or more embodiments.

FIG. 1E illustrates a schematic diagram including a portion of a second photolithography session in accordance with one or more embodiments.

FIG. 1F illustrates a schematic diagram including a portion of a second photolithography session and a tumor in accordance with one or more embodiments.

FIG. 2 illustrates a fluorescence image of circular constructs and surrounding matrix in accordance with one or more embodiments.

FIG. 3A illustrates a representative fluorescence images of a cell-embedded tumor model in accordance with one or more embodiments.

FIG. 3B illustrates a chart of cell viability where Data is presented in mean±SD, (Scale bars represent 200 μm) in accordance with one or more embodiments.

FIGS. 4A-4C illustrate representative phase contrast images demonstrating changes in cellular morphology in accordance with one or more embodiments.

FIG. 5A illustrates representative fluorescence images of migration and proliferation of the cells within the tumor model in accordance with one or more embodiments.

FIG. 5B illustrates a total cell count in accordance with one or more embodiments.

FIG. 5C illustrates a cell count within constructs in accordance with one or more embodiments.

FIG. 5D illustrates cell migration in accordance with one or more embodiments.

FIGS. 6A-6F illustrate images of cells in accordance with one or more embodiments.

FIG. 6G illustrates a chart of circularity amongst the three cell types (MCF10A, MCF7, and MDA-MB-231).

FIG. 7 illustrates phase contrast (3×3 tile) images of a high density array of tumor constructs demonstrating cellular morphology and migration. (Scale bars represent 250 μm), in accordance with one or more embodiments.

FIGS. 8A-8B illustrate phase contrast images of a control experiment using MDA-MB-231 cells in accordance with one or more embodiments.

FIGS. 9A-9B illustrate F-actin cytoskeletal organization of the cells demonstrating cells embedded within the hydrogel layer in accordance with one or more embodiments.

FIG. 10A illustrates representative cell tracks of MDA-MB-231 cells within the model. Blue lines indicate tracked cells that are initially within the circular construct at the start of the 12-h period, whereas orange tracks indicate cells that are initially outside the circular construct at the start of the 12-h period.

FIG. 10B illustrates reconstructed cell tracks normalized with respect to the origin.

FIG. 10C illustrates 360_{—} rose plots measuring normalized angular directionality. Scale bars represent 100 mm. n ¼ 156 cells.

FIG. 11A illustrates (Ai/ii) Scatterplot of velocity of MDA-MB-231 cells over time inside of the circular constructs and outside of the constructs, and (Aiii) comparative bar graph of cell velocities in the surrounding matrix versus the circular tumor construct in accordance with one or more embodiments.

FIG. 11B illustrates (Bi/ii) Scatterplot of persistence indices of cells over time, and (Biii) comparative bar graph of persistence. n ¼ 156 cells. Data is presented in mean±SD; * ¼ p<0.05.

FIGS. 12A-12I illustrate a-tubulin (red) and DAPI (blue) stained cell-embedded tumor model on day 5 of culture. (FIGS. 12A-C) are representative 40× Z-stack images of MDA-MB-231 cells, (FIGS. 12D-F) MCF7 cells, and (FIGS. 12G-I) MCF10A cells at three different angles (Top, Side, and 3D views).

### DETAILED DESCRIPTION

Breast cancer cell invasion is a highly orchestrated process driven by a myriad of complex microenvironmental stimuli. These complexities make it difficult to isolate and assess the effects of specific parameters including matrix stiffness and tumor architecture on disease progression. In this regard, morphologically accurate tumor models are becoming instrumental to performing fundamental studies on cancer cell invasion within well-controlled conditions.

A two-step photolithography technique is used to microengineer a 3D breast tumor model. The microfabrication process presented herein enabled precise control over the cellular distribution of the microenvironment and the creation of constructs adjacent to a surrounding matrix. A two-step photolithography technique and gelatin methacrylate (GelMA) hydrogel are used to develop a highly organized micropatterned breast tumor microenvironment model. GelMA has been proven to be an excellent candidate to generate biologically relevant constructs as cells have readily adhered to, proliferated within, and migrated when encapsulated within the 3D matrix of the hydrogel. More importantly, the use of GelMA enables the creation of arrays of specific cell-laden features with high precision and fidelity.

To validate the model, breast cancer cell lines (MDA-MB-231, MCF7) and normal mammary epithelial cells (MCF10A) were embedded separately within the tumor model and cellular proliferation, migration and cytoskeletal organization were assessed. Proliferation of metastatic MDA-MB-231 cells was significantly higher than tumorigenic MCF7 and normal mammary MCF10A cells. MDA-MB-231 exhibited highly migratory behavior and invaded the surrounding matrix, whereas MCF7 or MCF10A cells formed clusters that were confined within the micropatterned circular features. Our results indicate that gelatin methacrylate (GelMA) hydrogel, integrated with the two-step photolithography technique, assists in creating morphologically accurate 3D tumor models with well-defined features and tunable stiffness for detailed studies on cancer cell invasion and drug responsiveness.

A tumor model was created using a two-step photolithography technique and photocrosslinkable gelatin hydrogel. A unique aspect of our model was the compartmentalization of two distinct regions juxtaposed to each other with could potentially have differential stiffness and matrix composition. We validated the model by encapsulating three cell types separately in order to investigate migratory behavior, cell viability, and cell morphology. High viability was observed regardless of the cell type. Interestingly, a bimodal display of morphology was displayed in MDA-MB-231 cells as they elongated with flat protrusions on glass slid while exhibiting 3D protrusions or membrane blebs when invading the surrounding hydrogel matrix. These cells were highly populated at the high stiffness circular constructs. In addition, 3D cellular clusters were observed in both MCF7 and MCF10A cells. These morphologically accurate structures were formed without the use of any biochemical stimuli, which demonstrates the versatility of GelMA in creating a biomimetic tumor microenvironment. The proposed platform could be potentially used for future studies of cancer cell behavior, high-throughput drug screening, and the development of personalized medicine.

In one or more embodiments, a method for creating a tumor model includes encapsulating breast cancer cells in GelMA prepolymer solution, disposing the GelMA prepolymer solution on a spacer, disposing a photomask and glass slide on the spacer to form a first assembly, and exposing the first assembly to UV light to crosslink GelMA and creating an array of circular constructs on a first slide 110. The method further includes removing the first slide 110 from the spacer, disposing pristine GelMA prepolymer solution on to the spacer, disposing the first slide over the pristine GelMA and spreading the pristine GelMA prepolymer solution between the constructs to form a second assembly, and exposing the second assembly to UV light to crosslink the GelMA.

In one or more embodiments, exposing the second assembly and crosslinking the pristine GelMA includes creating a low stiffness matrix surrounding the high stiffness constructs. In one or more embodiments, the constructs could have a higher stiffness than the low stiffness matrix. In one or more embodiments, creating the array of circular constructs includes creating an array of circular constructs.

In one or more embodiments, a method for creating a tumor model includes conducting a first photolithography session including encapsulating breast cancer cells in GelMA prepolymer solution, disposing the GelMA prepolymer solution on a spacer, disposing a photomask and glass slide on the spacer to form a first assembly, exposing the first assembly to UV light to crosslink GelMA to create an array of circular constructs on a first slide, and removing the first slide from the spacer, and conducting a second photo lithography session including disposing pristine GelMA prepolymer solution on to the spacer, disposing the first slide over the pristine GelMA and spreading the pristine GelMA prepolymer solution between the circular constructs to form a second assembly, and exposing the second assembly to UV light to crosslink the GelMA.

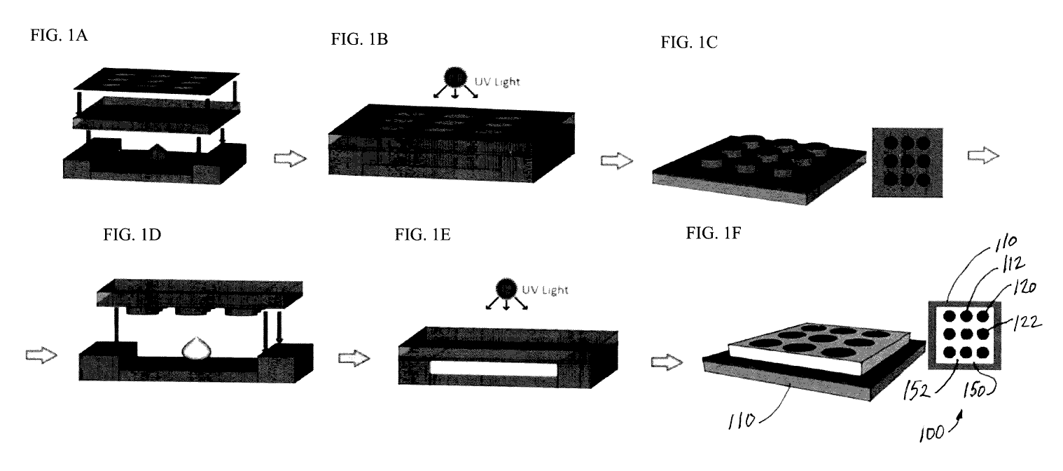

In one or more embodiments, the tumor model 100 includes a first slide 110 that has a first region 112 of high stiffness constructs 120 including cancer cells 122 encapsulated therein. The first slide includes a second region 152 of low stiffness matrix 150 that surrounds the high stiffness constructs, and the first region has a higher stiffness than the second region. (See FIG. 1).

In one or more embodiments, the tumor model and method are prepared as follows. For example, Gelatin Methacrylate (GelMA) hydrogel is prepared. Briefly, a 10% w/v solution of type A porcine skin gelatin was prepared in Dulbecco's phosphate buffered saline (DPBS; Gibco). This solution was made at 60° C. in order to fully dissolve before proceeding to subsequent steps. Methacrylic anhydride was then added drop-wise to infuse it within the gelatin solution. The mixture was then stirred vigorously for three hours as to ensure the completion of the reaction. In order to shift the equilibrium and stop the reaction, the reaction mixture was diluted (5×) with warm (40° C.) DPBS. This crude prepolymer GelMA was dialyzed for one week in distilled water (replaced twice a day) using dialysis membranes (MWCO 12000-14000) at a constant temperature (40° C.) to filter out any salt byproducts created from the reaction between gelatin and methacrylic anhydride. The desired degree of methacrylation was achieved by precisely controlling the proportion of methacrylic anhydride to gelatin during synthesis (92±2% confirmed based on 1H NMR). The gelatin methacrylate solution was lyophilized for one week to create a dehydrated, porous macromer, which could be preserved for future experiments.

### Cell Culture

The invasive breast cancer MDA-MB-231 cell line, non-invasive tumorigenic breast cancer MCF7 cell line, and normal mammary MCF10A cell line were used in this study. Cancer cells were maintained in 1×DMEM supplemented with 10% fetal bovine serum (FBS), 1% L-glutamine, and 1% 50:50 penicillin:streptomycin. Normal mammary cells were maintained in DMEM:F12 supplemented with 1% (w/v) L-glutamine, epidermal growth factor (20 ng/mL), cholera toxin (100 ng/mL), insulin (10 μg/mL), hydrocortisone (0.5 mg/mL), and 5% (w/v) horse serum. All media and media supplements were provided by Life Technologies. Cells were kept at a standard physiological condition (humidified, 37° C., 5% CO₂), were passaged weekly, and had their media changed every three days in order to produce a controlled experimental condition.

### Microfabrication of the Tumor Model

In order to promote adherence of the GelMA hydrogel constructs, glass slides were functionalized with 3-(trimethoxysilyl)propyl methacrylate (TMSPMA) (Sigma) as described in previous protocols. Subsequently, a 7 μL drop of 20% (w/v) polyethylene glycol (PEG) prepolymer solution included with 0.5% (w/v) photoinitiator (PI) (2-hydroxy-1-(4-(hydroxyethoxy)phenyl)-2-methyl-1-propanone) was placed onto cut (area: <1 cm²), sterilized glass slides. An untreated coverslip was placed on top of the PEG prepolymer and this arrangement was then exposed to ultraviolet (UV) light (360-480 nm, 800 mW) for 50 s which crosslinked to form a thin layer of PEG coating on the TMSPMA-treated glass slides.

To microengineer the tumor model, GelMA macromer was dissolved in DPBS containing 0.5% (w/v) PI. This formed a prepolymer solution, which was stored at 37° C. Cells were encapsulated in the prepolymer solution through resuspension of pelleted cells (cell density: 6×10⁶ cells per mL of GelMA). The tumor model was patterned by first pipetting a 15 μL droplet of cancer cell-laden GelMA onto a spacer (depth: 100 μm). A PEG-coated glass slide was then inverted on top of the spacer thereby spreading the prepolymer solution to cover the area of the glass slide and fill in the 100 μm depth of the spacer (see FIGS. 1A-1B). A photomask (designed with AutoCAD software and printed by CAD/Art Services Inc., Oregon) was then placed on the inverted, PEG-coated glass slide and exposed to UV light for 12 s (FIG. 1C). The photomask had a layout as described in Table 1 where a 11×11 array of translucent circles (radius: 250 μm) was surrounded by a black unpatterned area. Upon UV exposure, the patterned glass slide was washed to remove the excess cells and stored in a petri dish filled with DPBS. Following, a 13 μL drop of pristine GelMA (no cells) was placed onto the spacer and the patterned glass slide was inverted on top of it (FIG. 1D-1E). The circular constructs guided the spread of the pristine GelMA to the surrounding areas. This assembly was exposed to UV light for another 5 s in order to crosslink the gel filled in between the circular constructs (FIG. 1F). Upon completion, the micropatterned tumor model was transferred from the DPBS baths to 24-well cell culture plates with media corresponding to each cell line. Cell culture media was changed every three days.

### Cell Viability Assay

Cell viability was assessed on day 5 using a standard Live/Dead Assay Kit (Invitrogen), which includes calcein AM (CI) and ethidium homodimer (ETD). To prepare the solution, 0.5 μl CI and 2 μl ETD were added to 1 mL DPBS. After 5 days of culture, the microenvironments were rinsed with warm DPBS and 150 μl of the CI/ETD solution was added to each well. The well plate was stored at physiological conditions (37° C., humidified, 5% CO₂) and imaged after 30 minutes using an inverted fluorescence microscope (Zeiss Axio Observer Z1) with 10× magnification. Images were quantified by thresholding individual channels (red, green) and counting individual cells using the particle analyzer module of ImageJ. All cells were transfected, expressing red fluorescence. Percent viability was calculated by dividing the number of live cells by the total number of cells.

### Quantification of Cell Proliferation

Cell proliferation was quantified through counting cell nuclei on days 0, 1, 3 and 5 of culture. The cell-laden GelMA hydrogel constructs were rinsed with DPBS and fixed with 4% paraformaldehyde (PFA) solution in DPBS. After 30 minutes, the samples were washed three times (3×) in DPBS. A 0.1% (v/v) of DAPI (4′,6-diamidino-2-phenylindole) (Life Technologies) in DPBS solution was prepared and added to each well. The samples were left in DAPI contained solution for 15 minutes, and then washed 3× in DPBS. The samples were fluorescently imaged, and the number of DAPI stained nuclei were counted using ImageJ (v. 1.48) software to determine proliferation and migration of each cell line at specific time points (Days 0, 1, 3 and 5). At least three samples were prepared for each condition within each experiment.

### Actin Cytoskeletal Organization

In one or more embodiments, to assess F-actin cytoskeletal organization, cell encapsulated hydrogel constructs were fixed with 4% PFA solution in DPBS and then permeabilized for with 0.1% Triton X-100. The samples were washed 3× in DPBS with 5-minute intervals. The cell encapsulated hydrogel constructs were then blocked with 1% bovine serum albumin (BSA) for 1 hour. A 1/40 dilution of Alexa Fluor-488 phalloidin (Life Technologies) in 0.1% BSA was added to the blocked samples for 45 minutes. The hydrogel constructs were subsequently washed 3× in DPBS. Upon F-actin staining, the cells were stained with DAPI to visualize the nuclei. The stained samples were inverted onto a glass coverslip with a droplet of ProLong Diamond Antifade solution. The cell-encapsulated hydrogel constructs were imaged using a fluorescence microscope (Zeiss Axio Observer Z1) equipped with an Apotome.2 at 20×/40× magnification. Z-stacks and 2×2 tiles of the samples were obtained and 3D images were constructed using the Zen software. Circularity of the cells was determined by using top-view images of fluorescent F-actin staining. These images of individual constructs were fed into a custom script for the ImageJ software, which compared each individual clump or each individual cell to a perfect reference circle, outputting a percent circularity value.

In one or more embodiments, to assess F-actin and α-tubulin cytoskeletal organization, cell encapsulated hydrogel constructs were fixed with 4% (w/v) PFA solution in DPBS. The samples were rinsed 2× with DPBS-glycine for 10 min and washed with 0.05% (w/v) Tween-20 in DPBS for 10 min. The primary block, containing IF blocking solution (10% (w/v) goat serum, 0.2% (w/v) Triton X-100, and 0.1% (w/v) radioimmunoassay grade BSA) and 0.05% (w/v) Tween-20 in DPBS; was added to the samples for 1.5-2 h. Monoclonal mouse anti-α-tubulin (T9026, SigmaeAldrich) was diluted 1:500 (v/v) in IF buffer solution and centrifuged at 14 k RPM for 10 min. The samples were stained with this solution overnight at 4° C. and washed 3× in IF buffer with 20-min intervals between washes. A 1:200 (v/v) dilution of anti-mouse Alexa-Fluor® 555 in IF buffer solution was centrifuged at 14 k RPM to remove aggregates. The samples were then stained for 45 min and washed with IF buffer for 20 min. Subsequently, they were washed an additional 2× with 0.05% (w/v) Tween-20 in DPBS for 10 min. A 1:40 (v/v) dilution of Alexa Fluor-488 phalloidin (Life Technologies) and 1:1000 (v/v) dilution of DAPI in DPBS was added to the blocked samples overnight and incubated at 4 C. The hydrogel constructs were then washed 3× with 0.05% (w/v) Tween-20 in DPBS. Upon F-actin staining, the cells were stained with DAPI to visualize the nuclei. The stained samples were inverted onto a glass coverslip with a droplet of ProLong® Diamond Antifade solution. The cell-encapsulated hydrogel constructs were imaged using a fluorescence microscope (Zeiss Axio Observer Z1) equipped with an Apotome.2 at 20×/40× magnification. Z-stacks and 2×2 tiles of the samples were obtained and 3D images were constructed using the Zen software. Circularity of the cells was determined by using top-view images of fluorescent F-actin staining. The images of individual constructs were fed into a custom script for the ImageJ software, which compared each individual clump or each individual cell to a perfect reference circle, outputting a percent circularity value.

### Data Collection and Statistical Analysis

Migration and proliferation data were analyzed over the course of three experiments (n=3) for each cell line. Each experiment (sample) had three replicates for a total of nine replicates per cell line at each time point (Days 0, 1, 3, 5). The data was collected within a 5×5 array of constructs in the center of each replicate. Data for the live-dead analysis had the same method of data collection in terms of experiments, sample sizes, and replicates on day 5 of culture. Data for circularity was collected by measuring the circularity of the cells within the triplicate samples of one experiment for each of the three cell types.

A one-way analysis of variance (ANOVA) was conducted, which demonstrated statistically significant differences between each group when α=0.05. A Bonferroni's post-hoc test was subsequently completed in order to measure statistically significant differences between individual groups. All data were presented in mean±standard deviation (SD). Statistical analysis/data presentation were performed in Graph Pad Prism (v. 6.0).

### Microfabrication and Characterization of the Tumor Model

The microengineered tumor model was developed using 5% GelMA with high (92±2%) degree of methacrylation due to its biocompatibility and reliability for photolithography applications. The specific geometrical parameters of the microengineered tumor model are defined in Table 1.

TABLE 1
Geometrical features of the microengineered tumor model*

| | Depth | Diameter | Spacing | Surface ratio construct/ |
|---|---|---|---|---|
| Shape | (μm) | (μm) | (μm)** | surrounding |
| Circle | 100 | 500 | 750 | 0.536 |

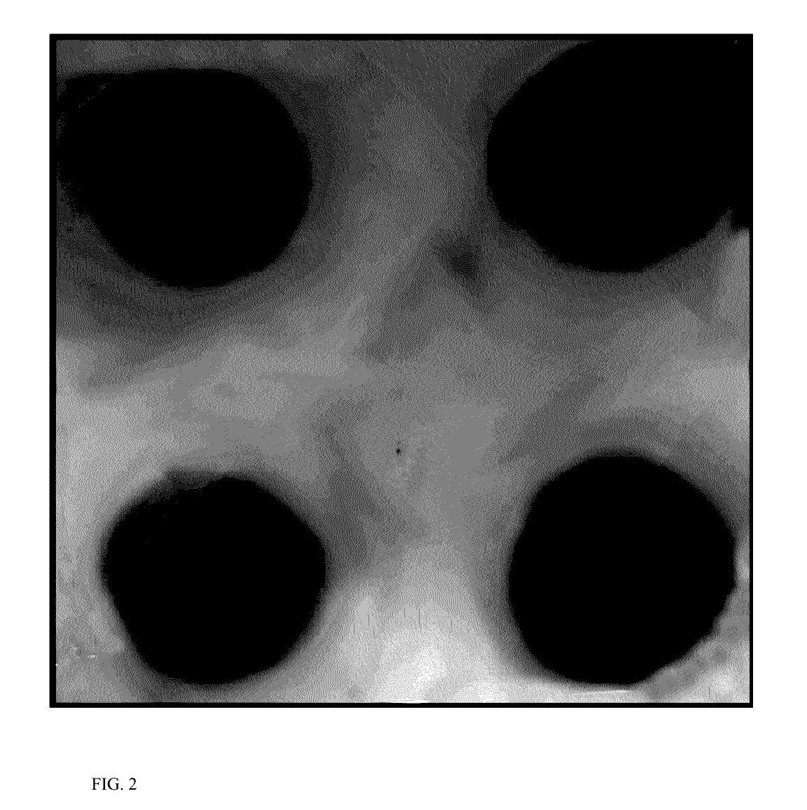

*Visualized in FIG. 2A which shows representative fluorescence image of Rhodamine B stained circular constructs and Fluorescein stained surrounding matrix.
**Spacing refers to the distance between the radii of adjacent tumor construct

The thickness of the tumor constructs was set to 100 μm due to its proven efficacy in the formation of patterned cellular constructs. The spacing and diameter of the cell encapsulated circular constructs were optimized based on a series of preliminary experiments (data not presented). In particular, circular constructs of smaller diameters (250 μm, 100 μm) had lower pattern precision, forming rough edges and unclear boundaries around the circular constructs. Conversely, 500 μm constructs exhibited pristine boundaries and smooth edges. However, the spacing between circular constructs had no real effect on the precision of the photolithography process and can be adjusted in future studies depending on throughput needs. The parameters defined in this study allowed for excellent pattern fidelity and analysis on a high-throughput scale with an 11×11 array of tumor constructs per chip.

Upon optimization, separate aliquots of GelMA prepolymer solution were stained with 0.01% rhodamine and 0.01% fluorescein dye to visualize the localization of hydrogel constructs after micropatterning. The developed two-step photolithography technique, as demonstrated in FIGS. 1A-1F, was used to form high density array of circular constructs (red stained hydrogel) surrounded by a surrounding matrix (green stained hydrogel). In particular, the two-step process involved fabricating the circular constructs first (FIG. 1A-C), and, subsequently, filling in the surrounding regions by adding GelMA prepolymer in between the constructs (FIG. 2).

Referring to FIGS. 1A-1F, the figures illustrate a schematic diagram depicting the development of array of the proposed tumor model. As shown in FIG. 1A, a drop of breast cancer cells encapsulated in GelMA prepolymer solution is pipetted onto a spacer and a glass slide/photomask is layered on top of it. Referring to FIGS. 1B and 1C, UV light is exposed to crosslink GelMA to create an array of high stiffness circular constructs. FIG. 1D illustrates a drop of pristine GelMA prepolymer solution is being pipetted onto a spacer and the micropatterned circular constructs from FIG. 1C is placed on top of it, thereby spreading the hydrogel in between the constructs. FIG. 1E shows that UV light is exposed to crosslink the surrounding matrix. FIG. 1F illustrates a representative schematic of the final microengineered tumor model with the high stiffness tumor constructs surrounded by low stiffness matrix.

The circular constructs were, as such, crosslinked more than the surrounding matrix. As the crosslinking time of the prepolymer solution has a direct positive correlation to the stiffness of the GelMA hydrogel, we expected that this method would create cell-embedded circular constructs with stiffness that is substantially higher than the surrounding matrix assess the capability of the proposed microfabrication technique in forming areas of differential stiffness or composition on a single chip.

### Cell Viability

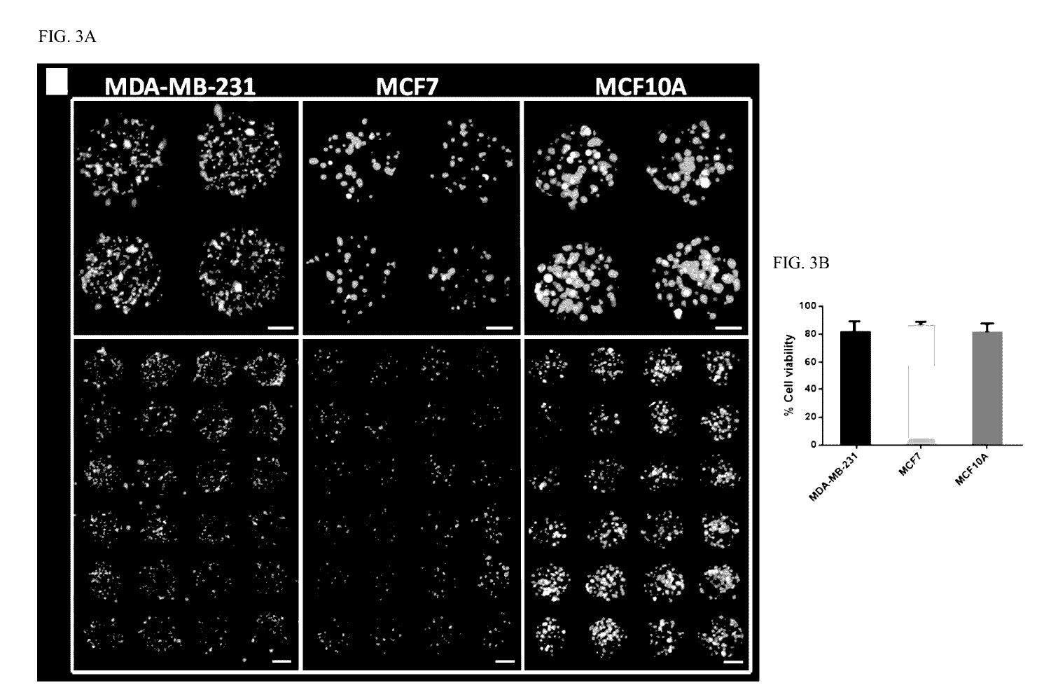

We evaluated viability of three distinct cell types, non-tumorigenic mammary epithelial MCF10A cells, non-invasive tumorigenic MCF7 cells, and highly invasive breast cancer MDA-MB-231 cells encapsulated within the microengineered tumor model on days 1 and 5 of culture. Representative images of the cell viability experiments (FIG. 3A) demonstrated excellent cell survival upon encapsulation and the microfabrication procedure. The percent of viable cells across all the three cell types had no statistically significant difference and was within 92±3% after one day of encapsulation, which decreased to 83±5% after 5 days of culture (FIG. 3B). Similarly, in previous studies, a wide array of other cell types such as ovarian cancer cells, 3T3 fibroblast cells, cardiomyocytes, and HUVECs, encapsulated within GelMA hydrogel, exhibited high percent cell survival upon micropatterning. Thus, our data confirmed that the specific parameters used to microengineer the tumor model (the two-step, 17 second UV exposure and presence of PI within the prepolymer solution) did not have a substantial effect on overall cell viability.

Cell Morphology, Migration, and Proliferation within the Micropatterned Constructs

Phase contrast images demonstrated that the three cell types (MCF10A, MCF7, MDA-MB-213) were homogeneously distributed throughout the hydrogel and had a round morphology on day 0 immediately after encapsulation within the micropatterned circular regions. However, between days 1 and 3 of culture, the cells began to exhibit characteristics specific to the cell type.

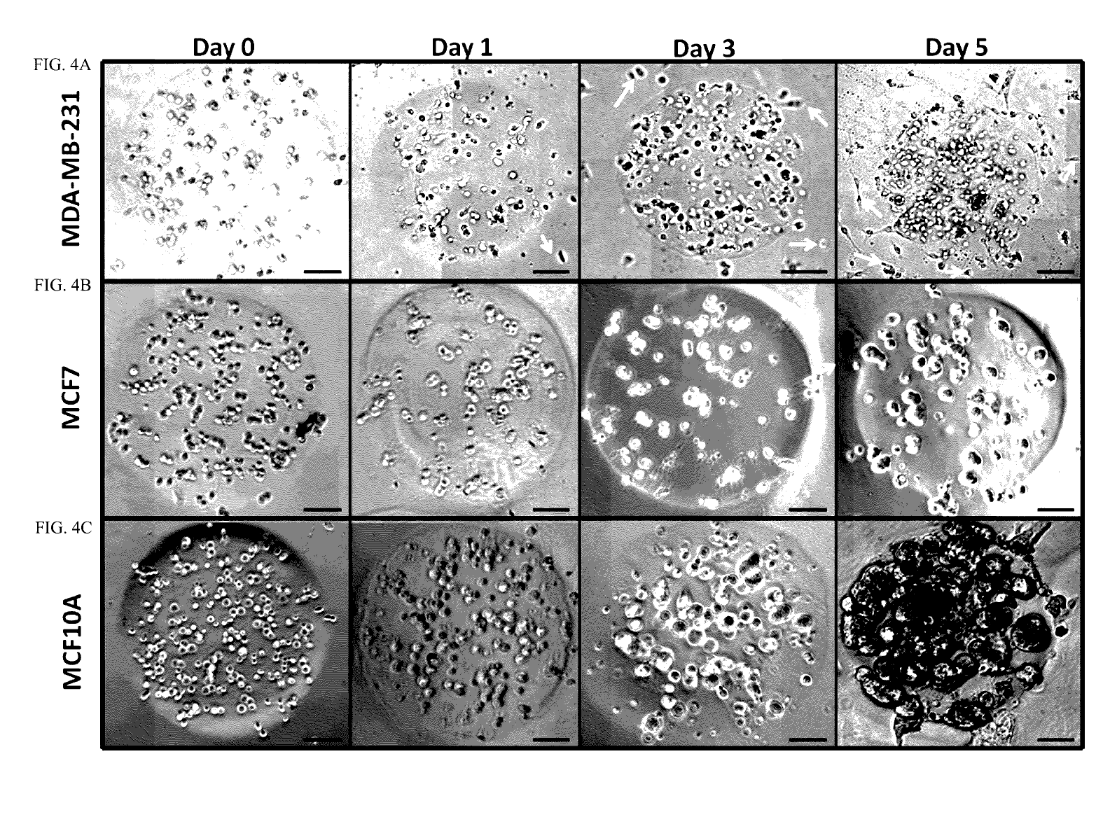

Referring to FIGS. 4A-4B, representative phase contrast images demonstrating changes in cellular morphology. MDA-MB-231 cells spread rapidly creating a heterogeneous (spindle vs. round) morphology. Arrows point to cells that have invaded the surrounding stroma. MCF7 cells exhibited a tendency to cluster, demonstrating only weak migration on days 1 and 3 of culture and small clusters by day 5. MCF10A cells formed similar clusters by day 3 which grew bigger by day 5. Scale bars represent 100 μm.

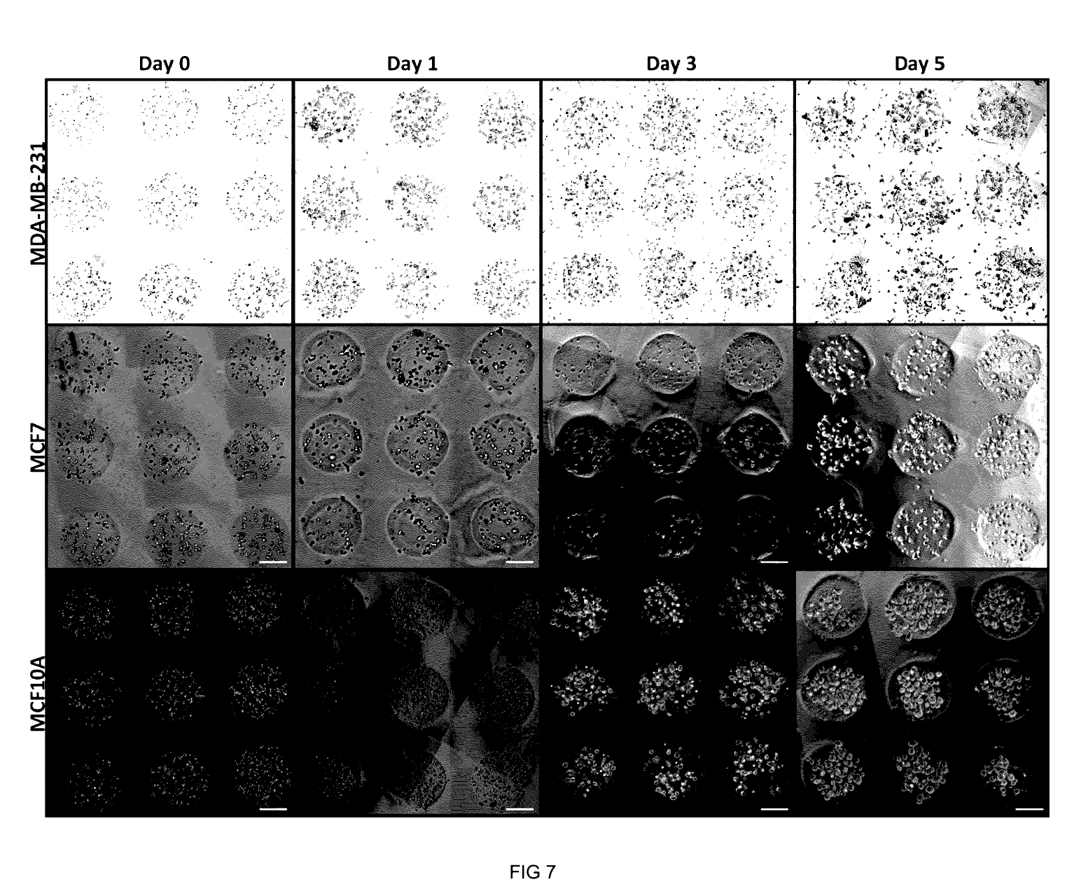

MDA-MB-231 cells adopted a heterogeneous morphology, both round and elongated, with higher cell density secondary to their high proliferative capacity (FIG. 4A). These cells started migrating toward the outer regions of the circular constructs as early as day 3 of culture, which was further evident on day 5 of culture (Arrows, FIG. 4A; FIG. 7. On the other hand, MCF7 cells formed clusters within and on the periphery of the circular constructs and exhibited weak migratory characteristics and elongation toward the surrounding regions as early as day 1 of culture (Arrows, FIG. 4B). These cells had no indication of an invasive phenotype by day 5 as they lost their elongated morphologies and quickly began to form clusters (FIG. 4B). Similarly, MCF10A cells also formed cellular clusters upon day 1 of culture and demonstrated no significant migratory characteristics (FIG. 4C). These cells maintained round morphology, while the size of the cellular clusters notably increased as a function of time.

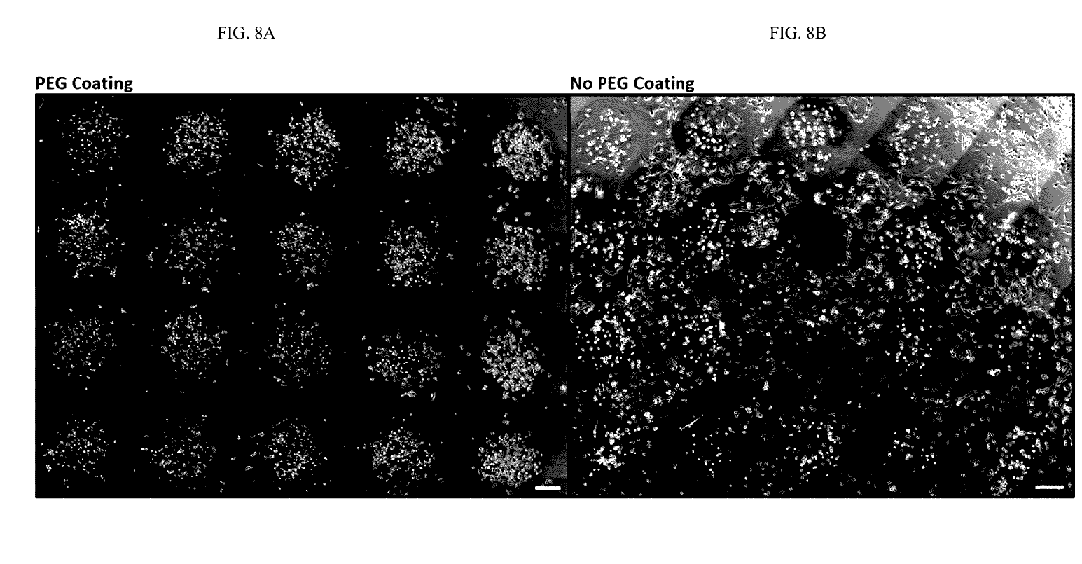

To prevent cellular attachment onto the glass slide and promote the cellular migration throughout the 3D hydrogel constructs, a layer of PEG was coated onto the glass slide due to its cell-repellant properties. Control experiments were conducted where the circular constructs were patterned onto glass slides with and without PEG coating. When patterned on slides without PEG, nearly every single cell escaped from the micropatterned circular regions and migrated onto the glass slide (FIG. 8, which show phase contrast images of a control experiment using MDA-MB-231 cells). These results indicate that, without PEG, the cells heavily adhered to and interfaced with the glass slide. On the other hand, adding PEG coating resulted in cell-repelling properties and facilitated the migration of the cells throughout the hydrogel layer. In the presence of PEG, cells were confined within the circular constructs at all time points. Without PEG coating, cells migrated down to the glass slide before diffusely migrating on the glass slide. Scale bars represent 200 μm.

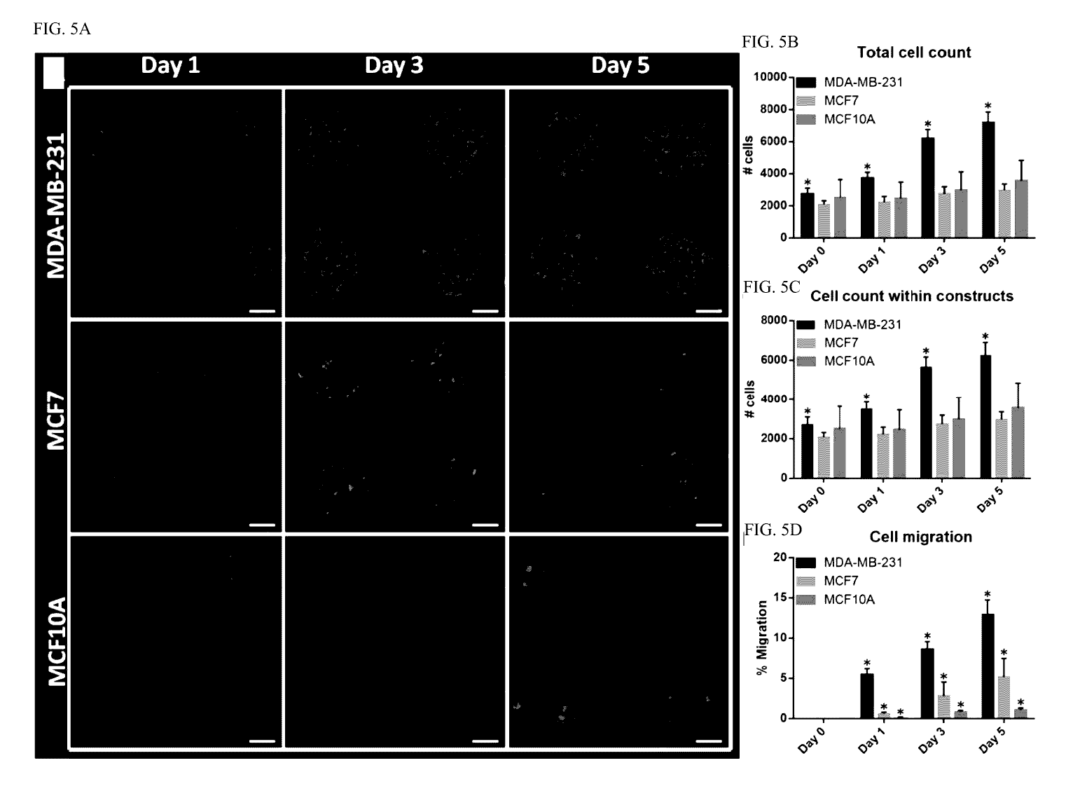

Consistent with phase contrast images, fluorescence images of DAPI stained cell nuclei demonstrated a significantly higher number of MDA-MB-231 cells within the circular constructs and the surrounding matrix as compared to MCF7 and MCF10A cells. Referring to FIGS. 5A-5D, representative fluorescence images are shown in FIG. 5A demonstrating DAPI stained cell nuclei, total cell proliferation FIG. 5B, cell proliferation within the tumor region FIG. 5C, and cellular invasion FIG. 5D. Scale bars represent 200 μm. *p<0.05 compared to the previous time point.

Cellular clustering was also evident in DAPI stained MCF7 and MCF10A cells (FIG. 5A). Quantitative analyses confirmed that the overall MDA-MB-231 proliferation was significantly higher compared to MCF7 and MCF10A cells within the microengineered platform (FIG. 5B). Particularly, a similar trend was observed with respect to the number of the cells within the circular constructs (FIG. 5C). About 2.5 times more MDA-MB-231 cells disseminated from the circular areas toward the surrounding matrix by day 5 of culture as compared to MCF7 cells (12.9±1.9% vs 5.2±2.301%). MCF10A cells exhibited nearly no invasive characteristics toward the outer regions of circular constructs (1.1±0.2% by day 5) (FIG. 5D). However, there was still a statistically significant difference in the migration of MCF10A cells at each time point. This is due to the clumping tendency as some cells proliferated to form clusters on the edge or boundaries of the constructs and the surrounding matrix. MDA-MB-231 cells were shown to elongate at the periphery of the constructs prior to contractile motion, which guided them out of the constructs. These cells demonstrated the ability to migrate between and back into constructs after initially invading the surrounding matrix. Consequently, migration data presented consists of net migration values counting only the cells that have entered and remain in the surrounding matrix by day 5 of culture. Alternatively, MCF7 and MCF10A proliferated to form large cellular clusters. While proliferating, cellular clusters were often seen rotating within the 3D matrix.

### Live Cell Tracking

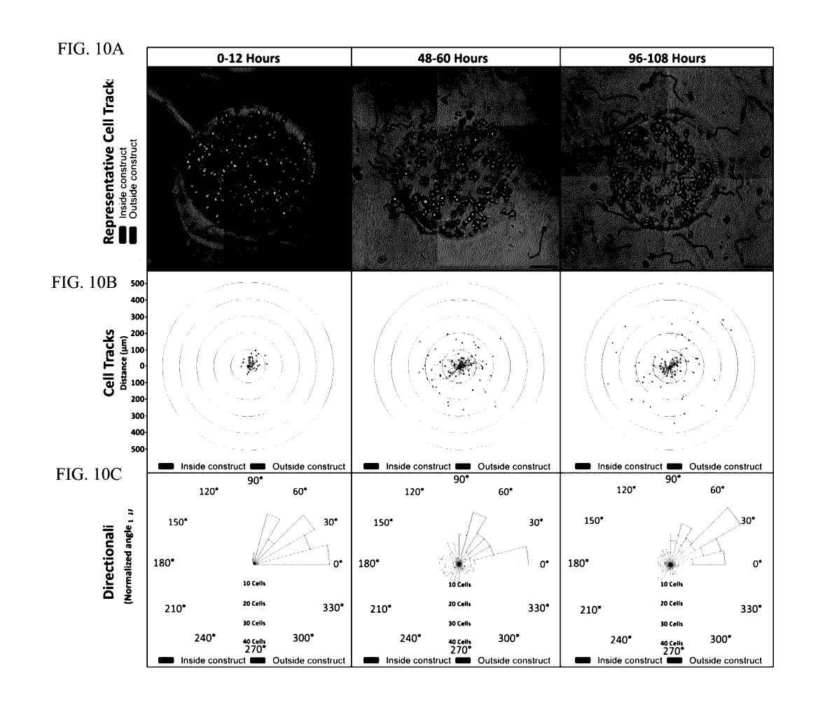

Due to the heterogeneous morphology of the MDA-MB-231 cells, we performed live cell tracking analyses to further investigate the migratory behavior induced by the architecture and biophysical properties of the tumor model. FIG. 10A, 10B demonstrate representative cell tracks of MDA-MB-231 cells within the model for 12-h time periods during each day of culture. The cells disseminated from the circular constructs and invaded the surrounding matrix during all time periods. Interestingly, there was a substantial difference in the migratory phenotype of the cells tracked inside the circular constructs compared to those tracked within the surrounding matrix. We specifically analyzed angular directionality and plotted it as a normalized 360_{—} rose plot (FIG. 10C). Cells inside the circular tumor region typically migrated in one common direction with respect to neighboring cells, while those in the surrounding matrix migrated with no particular (i.e. random) directionality.

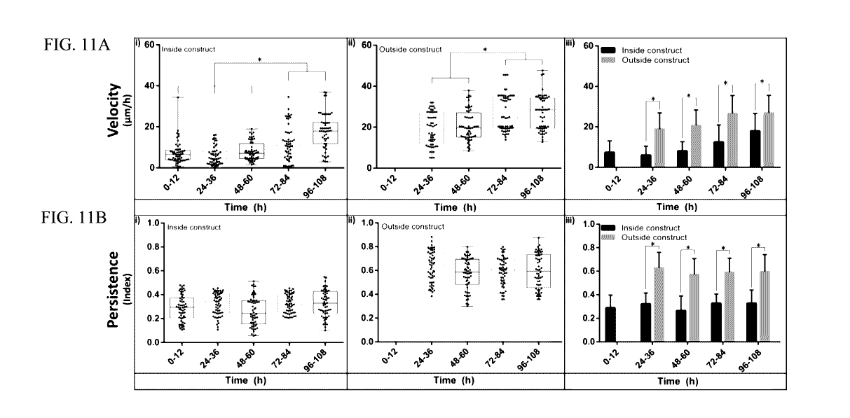

Additionally, we analyzed the velocity and persistence of the cells inside and outside of the circular constructs. Generally, migration speed increased as a function of time, and the cells slowly matured to adopt an invasive phenotype and morphology (FIG. 11A). The velocity of cells in the surrounding matrix was significantly higher than that of cells within the circular region. Alternatively, cells both inside and outside of the constructs did not have substantial changes in persistence as a function of time (FIG. 11B). However, cells migrating through the surrounding matrix exhibited a significantly higher persistence than cells inside of the circular constructs at all time points.

### Actin Cytoskeletal Organization

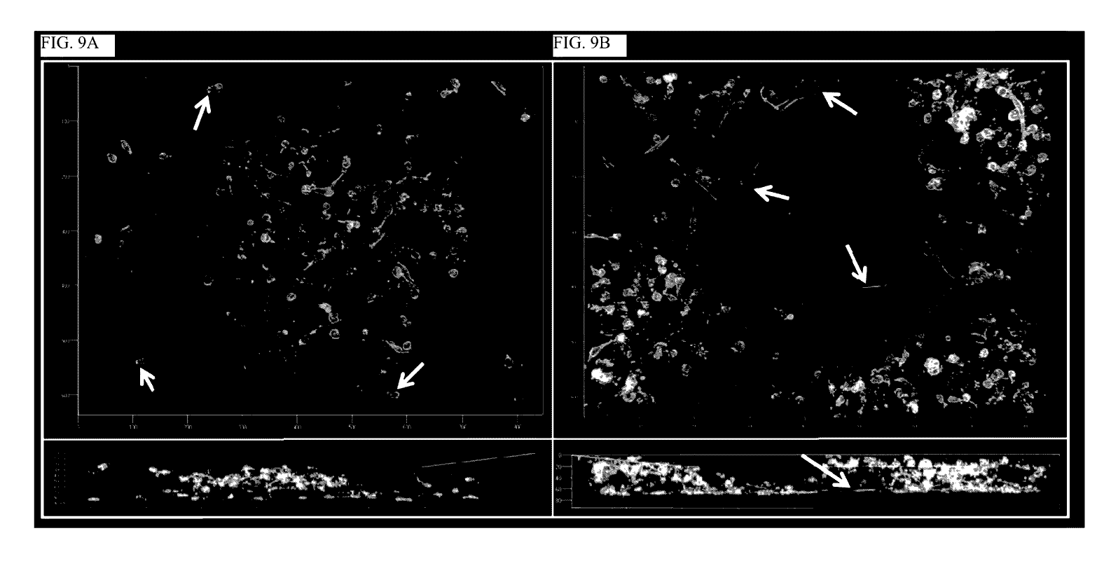

To further confirm our observations on cellular migration and gain insight into cell-matrix interactions/morphology, we performed 3D imaging of the actin cytoskeletal organization of cells embedded throughout the hydrogel layer (100 μm height). Preliminary images clearly demonstrated the cells were embedded within the hydrogel layer of the circular constructs (FIG. 9A) as well as the surrounding matrix (FIG. 9B). FIGS. 9A and 9B illustrate F-actin cytoskeletal organization of the cells demonstrating cells embedded within the hydrogel layer in accordance with one or more embodiments. Some MDA-MB-231 cells migrated to the glass slide and demonstrate a flat protrusions. These cells also exhibited 3D actin protrusions and membrane blebs.

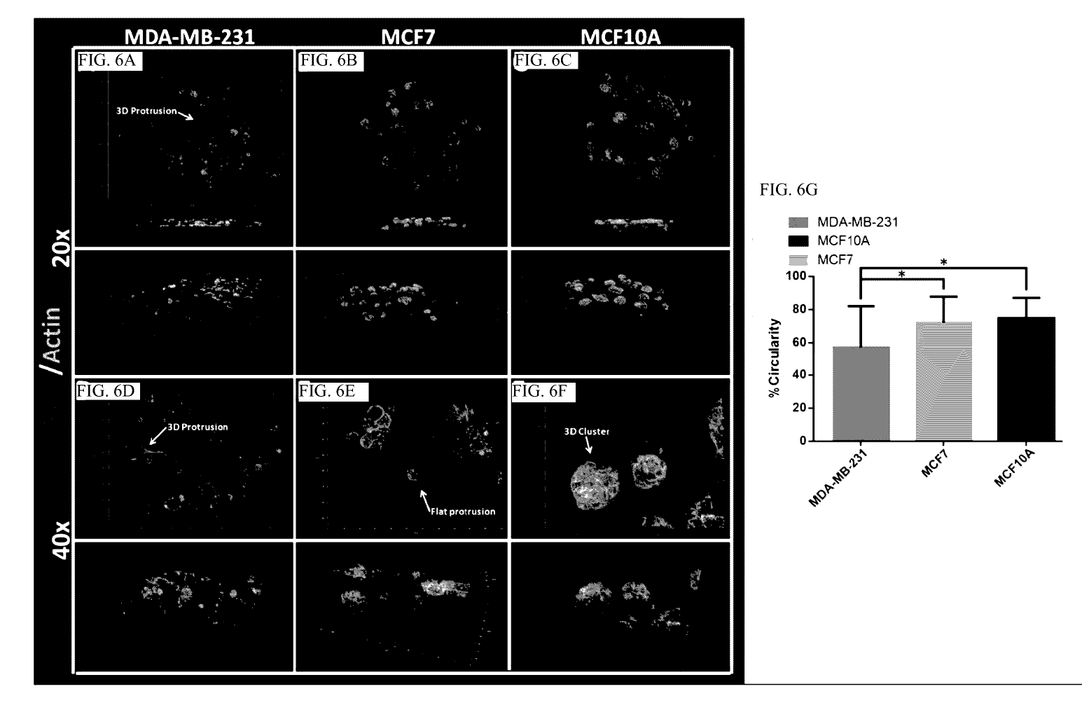

Using Z-stack immunofluorescence imaging of the actin cytoskeleton, we were able to visualize the 3D structure of the cells. FIGS. 6A-F illustrate F-actin (green) and DAPI (blue) stained cell-embedded tumor model on day 5 of culture, where FIGS. 6A-6C are representative 20× images of MDA-MB-231, MCF7, and MCF10A cells respectively, and FIGS. 6D-6F are representative 40× images highlighting specific cell-matrix interactions.

We observed several different structures including 3D elongated protrusions, flat protrusions and membrane blebs (FIGS. 6A-6F, arrows). In the representative images of the F-actin cytoskeleton, MDA-MB-231 cells particularly exhibited a wide range of invasive characteristics possessing small number of flat protrusions and many elongated 3D protrusions. In addition some cells exhibited membrane blebs as they invaded the surrounding matrix (FIG. 6A, D). A few number of MCF7 cells exhibited flat protrusions, on the periphery of the circular constructs, as demonstrated in the representative high magnification (40×) images (FIG. 6E). In MCF10A cells, the clustering tendency was significantly higher with no indications of protrusions.

To further quantify cellular morphology, the circularity of the actin cytoskeleton was assessed within the three cell types (FIG. 6G) using a custom script for Image J software (particle analyzer module). This analysis revealed that MDA-MB-231 cells exhibited a significantly less circular morphology when compared to MCF7 and MCF10A cells, as demonstrated by the high standard deviation indicative of their heterogeneous morphology (FIG. 6G). MCF10A, MCF7, and MDA-MB-231 cells had circularities of 74.9±12.1%, 72.1±15.7%, and 57.3±24.7% respectively (*p<0.05).

The development of 3D in vitro breast tumor models is significant for cancer related studies, since it would enable us to perform fundamental biological analyses on metastatic processes, such as cancer cell invasion. Furthermore, biomimetic tumor models can facilitate high throughput analyses on the efficacy of various pharmaceuticals compounds on cancer cell invasion. Currently, a wide variety of 2D and 3D platforms are being used to study breast cancer cell behavior (i.e. migration, gene expression). 2D assays do not recapitulate the complexities of the native tumor microenvironment. On the other hand, the majority of 3D hydrogel-based matrices lack organized architecture and cellular constructs, thus are limited in terms of localizing the stromal components and cancer cells within separate regions.

It is now becoming more recognized that the integration of microfabrication techniques and advanced biomaterials (i.e. photocrosslinkable hydrogels) can provide a unique ability to develop highly organized cell-based constructs. In this regard, GelMA hydrogel is an excellent candidate for cancer related studies due to its biocompatibility and ability to create organized cellular constructs. However, the primary focus on the use of GelMA, thus far, has been centered on tissue engineering and regenerative medicine applications (e.g. formation of vascularized networks). To our knowledge, there has not been any specific study utilizing GelMA to develop microengineered breast tumor models. Furthermore, there have been no significant attempts, using hydrogel-based matrices, to localize the separate regions with tunable stiffness (i.e. circular constructs, surrounding region) within microengineered platforms. In this work, we build upon our expertise in microfabrication technology by creating a novel, two-step photolithography technique to develop a 3D highly organized breast tumor microenvironment. GelMA has been demonstrated to be a biocompatible matrix for encapsulation with a vast array of cell types including 3T3 fibroblasts, endothelial cells, aortic valvular interstitial cells, and glioma cells. Consistent with previous studies, our work also confirmed that breast cancer and mammary epithelial cells had around 93% viability, which decreased to about 82% by day 5 of culture, indicating that the two-step photolithography technique along with the UV exposure and the presence of a PI had minimal effect on overall cell survival.

An innovative aspect of our study was independently patterning 3D high stiffness circular constructs surrounded by an interstitial area of lower stiffness (surrounding regions). Matrix stiffness demonstrates a physiologically relevant condition of the tumor microenvironment and has consequently been heavily studied in collagen, polyacrylamide, and Matrigel hydrogels. Furthermore, several studies have focused on seeding the cells on hydrogel sheets with different stiffness rather than encapsulating them within the 3D matrix, which may not accurately represent physiological cell behavior. In this regard, our model provides a distinct advantage, as we are able to independently modulate the stiffness of the matrix within distinguished regions in the microengineered tumor model. As such, we can assess the specific effects of matrix stiffness on breast carcinoma progression in vitro within a 3D model. In our model, it was demonstrated that some MDA-MB-231 cells were highly populated in the higher stiffness circular constructs. Although significant number of cells initially invaded within the surrounding regions of lower stiffness, but real time analysis demonstrated that some cells gained an affinity to move back into the high stiffness circular areas. Such behavior indicates the tendency of cancer cells to migrate within the stiffer regions. In one or more embodiments, switching the stiffness of the circular constructs and the surrounding matrix can be conducted using the microengineering technique described herein.

The proposed micropatterned tumor model also shed unique insight on cancer cell morphology. MDA-MB-231 cells adopted highly invasive characteristics with a mixture of round and spindle like morphologies. Specifically, the cells that migrated down on the glass slide formed flat protrusions, which was substantially different than the morphology exhibited by the cells embedded within the 3D gel (Arrows, FIG. 9B). This bi-modal display of migratory morphology demonstrates that the mechanism for MDA-MB-231 cells migration was heavily influenced by substrate interactions (2D vs 3D). Particularly, cells migrating through the hydrogel formed 3D protrusions or membrane blebs (Arrows, Supplementary FIG. 9A). These observations were consistent with the heterogeneous, 3D morphology of migrating cells cultured in Cell Derived Matrix (CDM) and Matrigel. In order to fully guide the migration of the cells through the 3D hydrogel gel, further modifications (i.e. concentration of PEG,) are required to further enhance cellular repellency of PEG layer. MCF7 and MCF10A cells rapidly clustered as early as day 2/3 that only grew bigger by day 5. In fact, there are numerous studies that have utilized various biochemical signals (i.e Cyclic AMP) or the co-cultures with cancer associated fibroblasts (CAFs) in order to produce stimulate growth of morphologically accurate cellular clusters similar to acinar structures. Within GelMA hydrogel, these cells formed clustered without the need for any biochemical stimuli, which further validates our model by confirming that it can readily recreate in vivo like morphologies.

The proposed photocrosslinkable hydrogel along with the two-step photolithography technique can be used to create tumor microenvironment models that have significant applicability in terms of modeling a physiologically relevant diseased condition. Specifically, matrix stiffness can be modified, cellular composition and organization can be tweaked, and biochemical stimuli can be added to the environment in an organized manner. The microenvironment remains to be a high-density, quantifiable, and morphologically accurate model regardless of the study. This has significant applicability in terms of high-throughput drug testing, the development of personalized medicine, as well as in fundamental studies of cancer biology. In the future, we plan to build upon this microenvironment by conducting detailed studies on effects of matrix stiffness on migration/morphology and the introduction of stromal components within the tumor model.

The two-step photolithography technique is unique in light of the creation of the extracellular matrix by crosslinking the cell-laden tumor constructs prior to filling pristine GelMA hydrogel in between the constructs. In doing so, we are able to independently decouple the cell-embedded regions from the surrounding matrix and create organized patterns with high precision and consistency. Furthermore, the tumor model allows for a quantifiable assessment of cellular migration with conditions that can be rigorously controlled.

In this study, we explore the use of a novel, two-step photolithography technique and gelatin methacrylate (GelMA) hydrogel to develop a highly organized, 3D, micropatterned breast tumor microenvironment model. GelMA has been proven to be an excellent candidate to generate biologically relevant constructs as cells have readily adhered to, proliferated within, and migrated when encapsulated within the 3D matrix of the hydrogel. More importantly, the use of GelMA enables the creation of arrays of specific cell-laden features with high precision and fidelity. The proposed platform, presented herein, has unique advantages through the ability to independently decouple different cell-embedded regions within the tumor model and independently tune their stiffness. Furthermore, the microfabricated model enables precise visualization of cancer cell migration within a 3D matrix in response to microenvironmental cues.

The methods and models herein have potential as a tumor model for cell migration studies. It can be utilized in a laboratory setting for studies of tumor biology, in the pharmaceutical industry for high throughput drug testing, and in a clinical setting for the development of personalized medicine. Available alternatives include high throughput spheroid assays to assess drug toxicity, transwell assays to determine cellular migration, and hydrogel based assays (matrigel, collagen, etc.) to determine cellular morphology and invasion.

Based on high-resolution live cell tracking analysis of the MDAMB-231 cells, we observed significant differences in directionality, persistence, and velocity, which were influenced by whether the cells were inside or outside of the circular constructs on the initial time point. FIG. 5C indicated that cells had a specific directionality on all days of culture when they were migrating within the circular constructs. This result was in line with previous studies, which demonstrated that cells leave tracks and realign matrices when moving through 3D hydrogels, resulting in collective migration. Thus, within our model, cells may have created tracks in the high stiffness circular constructs, which promoted migration in specific directions. Alternatively, the low stiffness surrounding matrix lacked structural support compared to the circular tumor region, so cells migrated with no particular angular directionality upon invasion.

Furthermore, the velocity of the cells moving through the surrounding matrix was substantially higher than those encapsulated within the tumor construct at all time points (FIG. 11A). This indicated that the stiffness of the 3D matrix may have played a role in migratory speeds. Other findings have also stated that cells exhibit an elongated, invasive phenotype within 3D hydrogels of lower stiffness. Cellular persistence indices (i.e. the sum of stepwise distances divided by the end-end distance) indicated that cells exhibited low persistence and consequently changed their paths frequently while migrating within the circular constructs. Furthermore, the high cellular density within the circular tumor constructs increased the possibility of cell collisions, which may have inhibited cell velocity and caused cells to frequently shift their paths (i.e. reduced persistence). This observation is consistent with previous studies on contact inhibition of locomotion (CIL), which suggested that populations of invasive cells consistently alter their paths to avoid collisions with one another. In future studies, we plan to perform further in-depth migration analysis by switching the stiffness to create low stiffness circular constructs with a high stiffness surrounding matrix. In addition, using the proposed model, we will be able to individually tune matrix stiffness and assess changes in cancer cell migration in response to a wide range of stiffness.

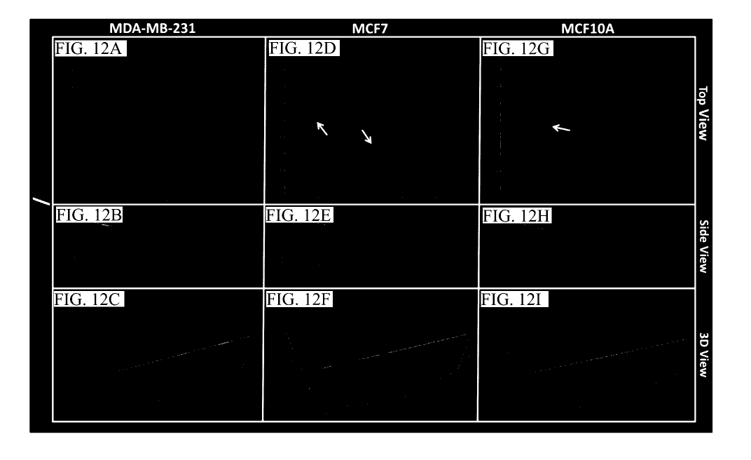

The proposed micropatterned tumor model also shed unique insight on cancer cell morphology. MDA-MB-231 cells adopted highly invasive characteristics with a mixture of round and spindle like morphologies. Specifically, the cells that migrated down on the glass slide formed flat protrusions, which was substantially different than the morphology exhibited by the cells embedded within the 3D gel (FIG. 6A, D; Arrows). This bimodal display of migratory morphology demonstrated that the mechanism for MDA-MB-231 cell migration was heavily influenced by substrate interactions (i.e. 2D vs 3D), in addition to matrix stiffness. Particularly, cells migrating through the hydrogel formed 3D protrusions and membrane blebs. These observations were consistent with the heterogeneous, 3D morphology observed in migrating cells cultured in Cell Derived Matrix (CDM) and Matrigel. Furthermore, due to the PEG coating on the glass slide of the proposed model, we observed that most of the cells were embedded within the 3D hydrogel matrix. In addition, MCF7 and MCF10A cells rapidly clustered as early as day 2 or 3, where the cluster grew larger by day 5. Interestingly, these cells adopted a different sheet-like morphology when proliferating on the periphery of the constructs. This was seen in phase contrast images and was also illustrated by 40× actin images. Cell morphology in our model was consistent with prior studies that have shown increased spheroid formation in high stiffness matrices. In fact, numerous studies have been conducted which required the utilization of various biochemical signals, such as Cyclic AMP or co-culturing cancer cells with cancer associated fibroblasts (CAFs), in order to stimulate growth of morphologically accurate cellular clusters. Thus, it is particularly interesting that when encapsulated within GelMA hydrogel, these cells readily clustered without the need for any biochemical stimuli. This further validates the ability of the proposed model to readily recreate in vivo-like morphologies. All three cell types demonstrated well-expressed microtubules throughout the 3D matrix (FIG. 12). Migrating MDA-MB-231 cells demonstrated small and large subcellular protrusions (FIG. 12A-C), while MCF7 and MCF10A cells (FIG. 12D-I) exhibited microtubule networks in spread cells and cellular clusters. Microtubules are of particular significance as they are indicative of biophysical stresses at the cell-matrix interface, which have rarely been studied within 3D micropatterned hydrogels. Additionally, various isotopes of microtubules are common targets of multiple drugs such as vinca alkaloids, taxanes, and nocodazole, which are common chemotherapeutics used today. In fact, there are numerous studies investigating the specific impact of such therapeutics on microtubule disruption. The ability of our model to assess potential changes in microtubule expression in a 3D matrix with well-defined architecture and tunable stiffness indicates that the proposed platform is a promising candidate to assess cell-matrix interactions in response to both biochemical and biophysical cues.

In this study, we created a tumor model using a novel, two-step photolithography technique and photocrosslinkable GelMA hydrogel. A unique aspect of our model was the compartmentalization of two distinct regions of the tumor microenvironment with differential stiffness. In particular, we developed high density array of cell embedded high stiffness circular constructs surrounded by a low stiffness matrix. We validated the model by encapsulating three cell types separately in order to investigate migratory behavior, cell viability, and cell morphology. High viability was observed regardless of the cell type. Interestingly, a bimodal display of morphology was displayed in MDA-MB-231 cells as, few cells migrated to the glass slide, elongating and assuming flat morphologies, while others exhibited 3D protrusions or membrane blebs when invading the surrounding hydrogel matrix. Due to the differential microarchitecture in our model with areas of distinct stiffness, cells migrating through the high stiffness circular constructs exhibited different invasive tendencies than those migrating through the surrounding matrix. In particular, cells in the surrounding areas migrated with high velocities and persistence when compared to those inside of the tumor constructs. We suspect that these behaviors were due to differences in matrix stiffness, as well as the high cell density within the patterned regions. In addition, 3D cellular clusters were observed in both MCF7 and MCF10A cells. These morphologically accurate structures formed without the addition of any biochemical stimuli, which demonstrates the versatility of GelMA in creating a biomimetic tumor microenvironment. The proposed platform could be potentially used for future studies of cancer cell behavior, high-throughput drug screening, and the development of personalized medicine.

In one or more embodiments, a method for creating a tumor model includes encapsulating cancer cells in a first solution, disposing the first solution on a spacer, cross-linking the first solution and creating one or more high stiffness constructs, disposing a second solution around the one or more high stiffness constructs, and cross-linking the second solution and creating a low stiffness matrix surrounding the one or more low stiffness constructs.

In one or more embodiments, encapsulating the cancer cells includes encapsulating breast cancer cells in GelMA prepolymer solution.

In one or more embodiments, cross-linking the first solution includes conducting a first photolithography session on the first solution.

In one or more embodiments, cross-linking the second solution includes conducting a second photolithography session on the second solution.

In one or more embodiments, disposing a second solution around the one or more high stiffness constructs includes disposing pristine GelMA prepolymer solution and spreading the pristine GelMA prepolymer solution between the high stiffness constructs.

In one or more embodiments, creating one or more high stiffness constructs includes creating an array of high stiffness constructs.

In one or more embodiments, cross-linking the first solution includes exposing the first solution to UV light.

In one or more embodiments, cross-linking the second solution includes exposing the second solution to UV light.

In one or more embodiments, creating one or more high stiffness constructs includes creating circular high stiffness constructs.

In one or more embodiments, a method includes conducting a first photolithography session including encapsulating breast cancer cells in GelMA prepolymer solution, disposing the GelMA prepolymer solution on a spacer, exposing the GelMA prepolymer solution and encapsulated breast cancer cells to UV light to crosslink GelMA and creating one or more high stiffness constructs on a first slide, and removing the first slide from the spacer; and

conducting a second photo lithography session including disposing pristine GelMA prepolymer solution on to the spacer, disposing the first slide over the pristine GelMA and spreading the pristine GelMA prepolymer solution between the high stiffness circular constructs, and exposing the second assembly to UV light to crosslink the GelMA.

In one or more embodiments, exposing the second assembly and crosslinking the pristine GelMA includes creating a low stiffness matrix surrounding the high stiffness constructs.

In one or more embodiments, creating one or more high stiffness constructs includes creating an array of high stiffness constructs.

In one or more embodiments, creating one or more high stiffness constructs includes creating circular high stiffness constructs.

In one or more embodiments, a method for creating a tumor model comprises encapsulating breast cancer cells in GelMA prepolymer solution, disposing the GelMA prepolymer solution on a spacer, disposing a photomask and glass slide on the spacer to form a first assembly, exposing the first assembly to UV light to crosslink GelMA and creating an array of high stiffness constructs on a first slide. The method further includes removing the first slide from the spacer, disposing pristine GelMA prepolymer solution on to the spacer, disposing the first slide over the pristine GelMA and spreading the pristine GelMA prepolymer solution between the high stiffness constructs to form a second assembly, and exposing the second assembly to UV light to crosslink the GelMA.

In one or more embodiments, exposing the second assembly and crosslinking the pristine GelMA includes creating a low stiffness matrix surrounding the high stiffness constructs.

In one or more embodiments, creating the array of high stiffness constructs includes creating an array of circular high stiffness constructs.

In one or more embodiments, a tumor model comprises a first slide having a first region of high stiffness constructs, the high stiffness constructs including cancer cells encapsulated therein, where the first slide includes a second region of low stiffness matrix surrounding the high stiffness constructs, and the first region has a higher stiffness than the second region.

In one or more embodiments, the high stiffness constructs are micropatterned circular constructs.

In one or more embodiments, the high stiffness constructs include crosslinked GelMA.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for creating a tumor model, the method comprising:
encapsulating cancer cells in a first solution;
disposing the first solution on a spacer;
cross-linking the first solution and creating one or more high stiffness constructs;
disposing a second solution around the one or more high stiffness constructs; and
cross-linking the second solution and creating a low stiffness matrix surrounding the one or more low stiffness constructs.

2. The method for creating a tumor model as recited in claim 1, wherein encapsulating the cancer cells includes encapsulating breast cancer cells in GelMA prepolymer solution.

3. The method for creating a tumor model as recited in claim 1, wherein cross-linking the first solution includes conducting a first photolithography session on the first solution.

4. The method for creating a tumor model as recited in claim 1, wherein cross-linking the second solution includes conducting a second photolithography session on the second solution.

5. The method for creating a tumor model as recited in claim 1, wherein disposing a second solution around the one or more high stiffness constructs includes disposing pristine GelMA prepolymer solution and spreading the pristine GelMA prepolymer solution between the high stiffness constructs.

6. The method for creating a tumor model as recited in claim 1, wherein creating one or more high stiffness constructs includes creating an array of high stiffness constructs.

7. The method for creating a tumor model as recited in claim 1, wherein cross-linking the first solution includes exposing the first solution to UV light.

8. The method for creating a tumor model as recited in claim 1, wherein cross-linking the second solution includes exposing the second solution to UV light.

9. The method for creating a tumor model as recited in claim 1, wherein creating one or more high stiffness constructs includes creating circular high stiffness constructs.

10. A method for creating a tumor model, the method comprising:
conducting a first photolithography session including encapsulating breast cancer cells in GelMA prepolymer solution, disposing the GelMA prepolymer solution on a spacer, exposing the GelMA prepolymer solution and encapsulated breast cancer cells to UV light to crosslink GelMA and creating one or more high stiffness constructs on a first slide, and removing the first slide from the spacer; and
conducting a second photo lithography session including disposing pristine GelMA prepolymer solution on to the spacer, disposing the first slide over the pristine GelMA and spreading the pristine GelMA prepolymer solution between the high stiffness circular constructs, and exposing the second assembly to UV light to crosslink the GelMA.

11. The method for creating a tumor model as recited in claim 10, wherein exposing the second assembly and crosslinking the pristine GelMA includes creating a low stiffness matrix surrounding the high stiffness constructs.

12. The method for creating a tumor model as recited in claim 10, wherein creating one or more high stiffness constructs includes creating an array of high stiffness constructs.

13. The method for creating a tumor model as recited in claim 10, wherein creating one or more high stiffness constructs includes creating circular high stiffness constructs.

14. A method for creating a tumor model, the method comprising:
encapsulating breast cancer cells in GelMA prepolymer solution;
disposing the GelMA prepolymer solution on a spacer;
disposing a photomask and glass slide on the spacer to form a first assembly;
exposing the first assembly to UV light to crosslink GelMA and creating an array of high stiffness constructs on a first slide;
removing the first slide from the spacer;
disposing pristine GelMA prepolymer solution on to the spacer;
disposing the first slide over the pristine GelMA and spreading the pristine GelMA prepolymer solution between the high stiffness constructs to form a second assembly; and
exposing the second assembly to UV light to crosslink the GelMA.

15. The method for creating a tumor model as recited in claim 14, wherein exposing the second assembly and crosslinking the pristine GelMA includes creating a low stiffness matrix surrounding the high stiffness constructs.

16. The method for creating a tumor model as recited in claim 14, wherein creating the array of high stiffness constructs includes creating an array of circular high stiffness constructs.

17. A tumor model comprising:
a first slide having a first region of high stiffness constructs, the high stiffness constructs including cancer cells encapsulated therein;
the first slide including a second region of low stiffness matrix surrounding the high stiffness constructs; and
the first region having higher stiffness than the second region.

18. The tumor model as recited in claim 17, wherein the high stiffness constructs are micropatterned circular constructs.

19. The tumor model as recited in claim 17, wherein the high stiffness constructs include crosslinked GelMA.
